(19) United States
(12) Patent Application Publication
(10) Pub. No.: US 2023/0187079 A1
(43) Pub. Date: Jun. 15, 2023

(54) SYSTEM AND METHOD FOR ASSESSING RISK PREDISPOSITION TO GESTATIONAL DIABETES AND DEVELOPING PERSONALIZED NUTRITION PLANS FOR USE DURING STAGES OF PRECONCEPTION, PREGNANCY, AND LACTATION/POSTPARTUM

(71) Applicant: Ali Mostashari, New York, NY (US)

(72) Inventors: Ali Mostashari, New York, NY (US); Raya Khanin, New York, NY (US); Klemo Vladimir, New York, NY (US); Mario Storga, New York, NY (US)

(73) Assignee: LifeNome Inc., New York, NY (US)

(21) Appl. No.: 18/073,551

(22) Filed: Dec. 1, 2022

Related U.S. Application Data
(60) Provisional application No. 63/287,751, filed on Dec. 9, 2021.

(51) Int. Cl. G16H 50/30; G16H 20/60

(52) U.S. Cl. CPC G16H 50/30; G16H 20/60

(57) ABSTRACT

A system for computing risk predisposition to gestational diabetes mellitus for an individual woman is provided. The system computes, based on received data, risk predisposition to gestational diabetes for a female. The system also calculates, based at least on the computed risk predisposition, calories and macro- and micro-nutrient needs for the female. The system also generates, based on the calculations, dietary recommendations, foods, and recipes for the female. The system further receives feedback from a plurality of female humans regarding liking/disliking and adverse reactions comprising at least one of morning sickness and nausea. The system further receives additional data comprising at least one of pregnancy complications, blood pressure, and glucose levels. The system further improves, based at least on the feedback and the received additional data, and via at least a machine learning methodology, assessment of risk predisposition to gestational diabetes for the plurality of female humans.

### CROSS REFERENCE TO RELATED APPLICATIONS

The present disclosure is related to U.S. Provisional Patent Application No. 63/287,751 filed Dec. 9, 2021, the contents of which are included herein in their entirety.

### FIELD OF THE DISCLOSURE

The present disclosure relates generally to systems and methods for assessing risk predisposition to gestational diabetes in women during stages of preconception, or early pregnancy, with objectives of mitigating the risk of gestational diabetes and improving maternal and child health through nutrition and lifestyle modifications. More particularly, the present disclosure provides systems and methods which utilize integrated individual women's genomics data (DNA, RNA) and non-genomics data and self-reported data and data from wearables to identify women with a higher risk of gestational diabetes and to personalize dietary recommendations and meal plans with the goal to minimize the risk of gestational diabetes.

### BACKGROUND

Gestational diabetes mellitus (GDM) is a common complication of pregnancy that adversely affects maternal and offspring health, GDM is characterized by the onset of hyperglycemia during pregnancy, typically in the second trimester, and is the most prevalent metabolic complication in pregnancy globally. Diagnostic criteria differ by region and are largely influenced by conventional care and the preferences of the clinicians. It is believed that 2%-5% of pregnancies worldwide are complicated with GDM, with the prevalence having significantly increased over the last decade. Globally, one in five pregnancies is affected by gestational diabetes. Furthermore, women who have experienced gestational diabetes have a ten-fold risk of developing type 2 diabetes later in life.

While the pathogenesis of the disease remains largely unknown, GDM is believed to be a result of interactions between genetic, genomic, epigenetic, and environmental factors. A variety of risk factors, such as body mass index (BMI) and advancing maternal age, have been associated with increased risk of GDM as well as other pregnancy complications. However, in many cases, GDM occurs in healthy nulliparous women with no obvious risk factors. Emerging data suggest that the tendency to develop pregnancy complications has a genetic component.

Multiple studies suggest that of chief importance among modifiable risk factors are physical activity and dietary intake before conception and during early pregnancy. The reduced level of physical activity during pregnancy is partly responsible for the pregnancy-associated decline in metabolic health.

Since modifying diet and lifestyle are key targets in the prevention and treatment of gestational diabetes, it is important to identify women who have a higher risk of developing gestational diabetes based on genomics and other factors and to offer such identified women actionable nutritional and lifestyle recommendations to minimize the risks. It is critical to identify these women either before or during the first trimester of their pregnancy.

Studies have shown that the actual implementation of lifestyle modifications reduces the risk of GDM. A systematic review has suggested that lifestyle intervention before the 15th gestational week may reduce GDM by 20%. A Randomized Controlled Trial demonstrated that moderate individualized lifestyle intervention reduced the incidence of GDM by 39% in high-risk pregnant women. (Altemani, A. H., Alzaheb, R. A. The prevention of gestational diabetes mellitus (The role of lifestyle): a meta-analysis. Diabetol Metab Syndr 14, 83 (2022). https://doi.org/10.1186/s13098-022-00854-5).

### PRIOR ART

Few disclosures to date address assessing the risk of Gestational Diabetes Mellitus (GDM) in women. WIPO PCT disclosure WO2017103106A1, entitled “Methods, tools and systems for the prediction and assessment of gestational diabetes,” provided risk assessment for GDM susceptibility based on a list of single nucleotide polymorphisms (SNPs). The disclosure only accounts for genetics without considering other factors such as age and BMI. The disclosure further does not provide a platform for actionable nutritional recommendations to women who have a higher risk of GDM. More important, while this disclosure constitutes an attempt to compute the DNA-based risk for GDM susceptibility, the SNP list is far from exhaustive, and the risk is not quantifiable.

Another previous disclosure that addresses GDM, European patent EP2836845A1 entitled “Maternal biomarkers for gestational diabetes,” is related to measuring proteomic biomarkers signatures to screen for GDM. Hence, the likelihood of GDM cannot be estimated before there are early measurable biomarkers. Furthermore, this disclosure does not provide preventative dietary and lifestyle recommendations to minimize the risk of GDM in the stage of preconception and in the early weeks of pregnancy.

Yet another disclosure, Chinese patent CN106446595B entitled “A kind of machine learning gestational diabetes mellitus risk and severity extent forecasting system,” uses the data of pregnant women, including the age, height, weight, blood glucose to predict the onset risk and/or severity extent of gestational diabetes. This invention does not utilize genetics and hence cannot estimate the likelihood of GDM before it occurs. It does not provide preventative dietary and lifestyle recommendations to minimize the risk of GDM.

Shortcomings, therefore, exist regarding systems and methods that assess the risks of GDM. The disclosed system and methods address the shortcomings of previous approaches and claim a dynamic self-learning system to identify women at high risk for GDM and provide them with personalized dietary and lifestyle recommendations to minimize the risk of GDM.

### BRIEF DESCRIPTION OF THE FIGURES

FIG. 1 is a block diagram of a system according to an embodiment of the present disclosure.

FIG. 1a is a block diagram of a system according to an embodiment of the present disclosure.

FIG. 2 is a block diagram of a system according to an embodiment of the present disclosure.

FIG. 2a is a block diagram of a system according to an embodiment of the present disclosure.

### DETAILED DESCRIPTION

Systems and methods provided herein identify women at risk of gestational diabetes mellitus (GDM) based on genomics, and other factors including biomarkers and demographics. Systems and methods provide personalized nutrition advice in the stages of preconception and early pregnancy. The advice is tailored to an individual woman's genetics, genomic data, and other considerations and may be critical to ensure the health and wellness of mothers and babies.

The present disclosure provides for collecting large amounts of heterogeneous data from pregnant women that can serve as a basis for longitudinal studies. A principal objective herein is to improve the assessment of risk predisposition to gestational diabetes and to further provide personalized nutrition and lifestyle recommendations that minimize the incidences of gestational diabetes.

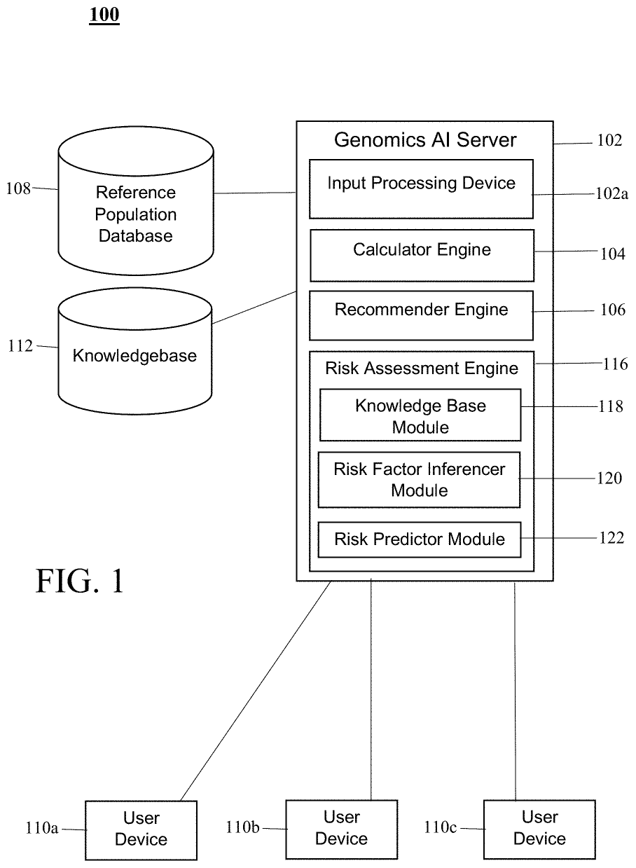

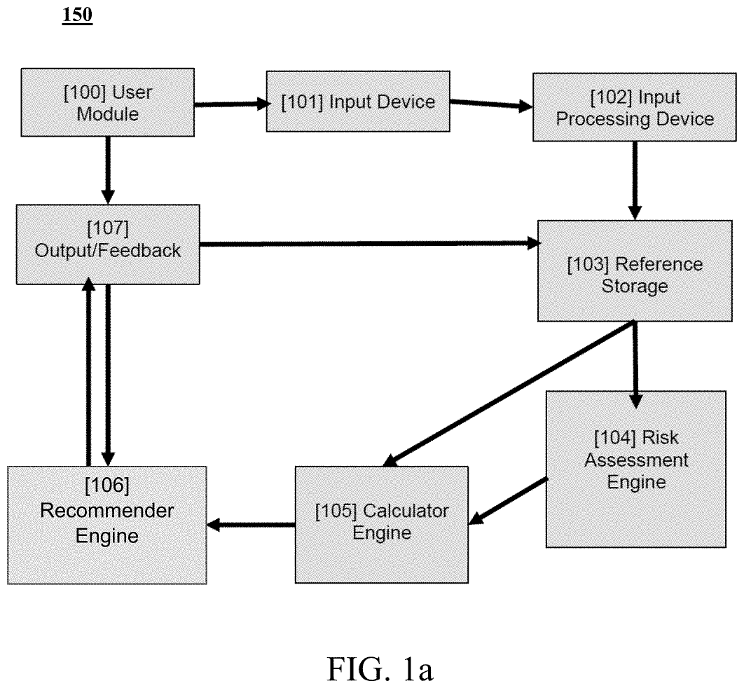

Turning to the figures, FIG. 1 illustrates the components and interactions of a system 100 for assessing risk predisposition to gestational diabetes and developing personalized nutrition plans for use during stages of preconception, pregnancy, and lactation/postpartum.

The system 100 comprises a Genomics AI server 102 that comprises a calculator engine 104, a recommender engine 106, and a reference population database 108. The system 100 also comprises a plurality of user devices 110a-c used by women to submit data to the Genomics AI server 102 and to receive dietary programs and other data from the Genomics AI server 102 and other components.

While quantity three user devices 110a-c are depicted in FIG. 1 and provided by the system 100, in embodiments more than or less than quantity three user devices 110a-c may be provided. The system 100 also comprises a knowledge base 112.

The system 100 also comprises a risk assessment engine 116. The risk assessment engine 116 comprises a knowledge base module 118, a risk factor inferencer module 120, and a risk predictor module 122.

The Genomics AI server 102 may be a single computer or multiple physical computers situated at one or multiple geographic locations. While the calculator engine 104, the recommender engine 106, and the risk assessment engine 116 are depicted in FIG. 1 as contained by or components of the Genomics AI server 102 and executing on the Genomics AI server 102, in embodiments the calculator engine 104, the recommender engine 106, and the risk assessment engine 116 may be separate components or software executing on separate devices proximate or remote from the Genomics AI server 102.

While referred to as engines, the calculator engine 104, the recommender engine 106, and the risk assessment engine 116 may be combinations of hardware and software applications or entirely software applications. Components described herein as modules, submodules, or devices may be physical devices, combinations of a physical device and software, or entirely software. For example, the knowledge base module 118, a risk factor inferencer module 120, and a risk predictor module 122 may be combinations of hardware and software or primarily software.

The Genomics AI server 102 receives genomics and non-genomics data from women using the user devices 110a-c. The received data is processed by an input processing device 102a of the Genomics AI server 102 and stored in the reference population database 108. The received data is also provided to the calculator engine 104 to determine macro- and micro-nutrient needs and ranges for the woman.

Based on the macro- and micro-nutrient needs and ranges determined by the calculator engine 104, the recommender engine 106 generates dietary recommendations, foods, and recipes. Feedback 17 regarding liking/disliking dietary recommendations and adverse effects such as morning sickness, or nausea, may be provided back to the recommender engine 106, the calculator engine 104, the reference population database 108, and the risk assessment engine 116 to improve future personalized dietary recommendations. The calculator engine 104 and the recommender engine 106 may rely on various algorithms to complete their tasks.

Additional data, such as the rate of weight gain during pregnancy, weight loss postpartum, blood pressure, glucose levels, and measurements of other biomarkers, may be collected from the woman and submitted to the components of the system 100 to improve future personalized dietary recommendations, and to discover relationships between genetics, genomics, dietary consumption, and preconception, pregnancy, lactation/postpartum traits.

FIG. 1a is a flow drawing depicting components of a system 150 according to an embodiment of the present disclosure. The components of the system 150 and depicted in FIG. 1a correspond to the components provided by the system 100 and depicted in FIG. 1. FIG. 1a illustrates the system 150 of receiving genomic and non-genomic data from a user, computing, via a machine learning methodology, risk predisposition to gestational diabetes for an individual woman, further calculating personalized calories, including macro- and micro-nutrient needs and ranges based on the received data, and generating personalized dietary and lifestyle recommendations.

As shown in FIG. 1a, an input device [101] is used to receive genomics and non-genomics data for females from a user module [100]. The received data is then processed by an input processing device [102] and added for storage into reference storage [103]. The received data is thereafter provided to a risk assessment engine [104] to compute risk predisposition to gestational diabetes for the individual female, and further calculate, via a calculator engine [105], personalized calories, macro- and micro-nutrient needs and ranges for the individual woman.

Based on the calculated calories, macro- and micro-nutrient needs, and ranges, a recommendation engine [106] generates dietary recommendations, foods, and recipes. Feedback regarding liking/disliking recommended foods and recipes, and adverse effects such as morning sickness and nausea may be provided back to the recommendation engine [106], the calculator engine [105], and the reference storage [103] to improve future recommendations.

Data on pregnancy complications, including gestational diabetes, pre-eclampsia, eclampsia, miscarriage, labor, birth details, and baby weight may be collected from the individual woman. Additional data, such as blood pressure, glucose levels, measurements of other biomarkers, rate of weight gain during pregnancy, or weight loss postpartum, may be collected from the individual woman and transmitted to the risk assessment engine [104], the calculator engine [105], and the reference storage [103].

These actions are intended to improve, via a machine learning methodology, the assessment of risk predisposition to gestational diabetes, as well as other pregnancy-related traits and complications, and to further improve future personalized dietary recommendations. Further, this longitudinal date is utilized to discover associations, and to build, via a machine learning methodology, predictive comprehensive models for pregnancy-related traits and pregnancy complications, taking into consideration genomics, and other data, including dietary consumption and lifestyle during preconception, pregnancy, and postpartum stages.

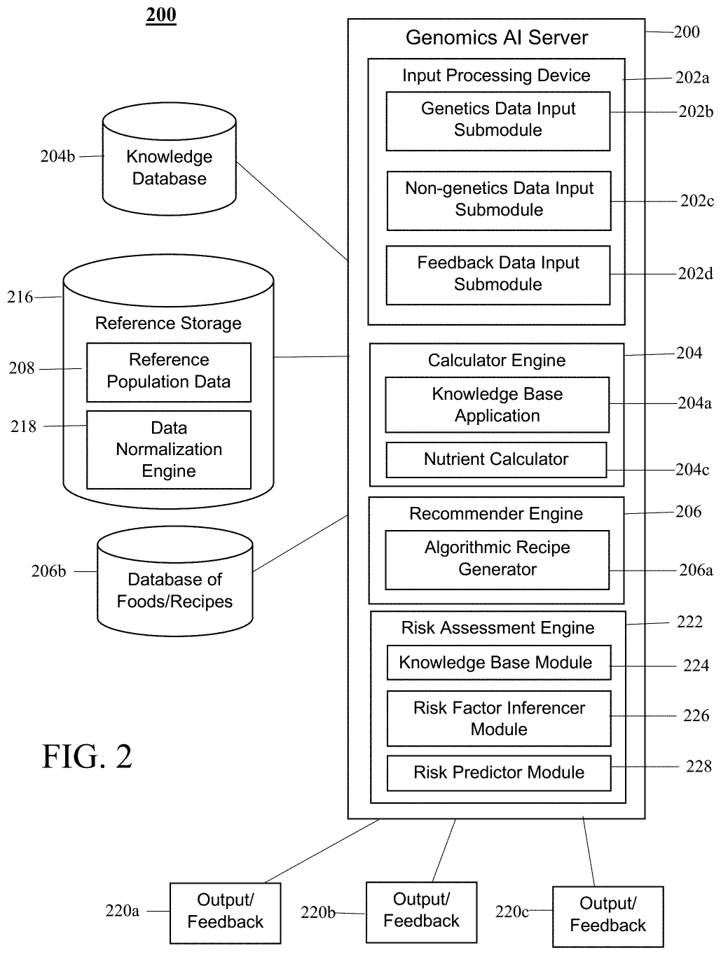

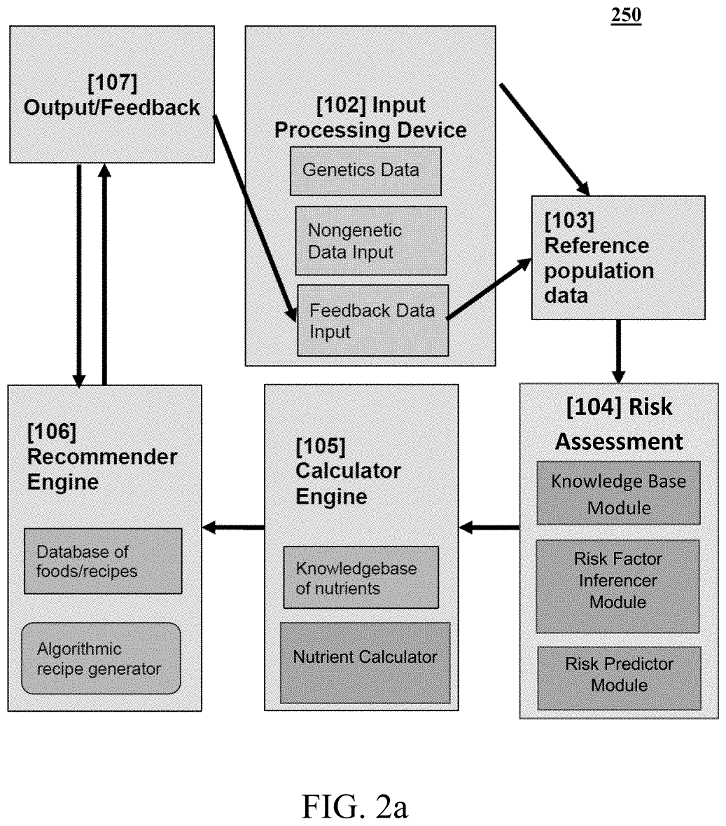

FIG. 2 is a block diagram depicting a system 200 provided by an embodiment of the present disclosure. Components of the system 200 are partially indexed to components of the system 100.

A Genomics AI server 202 is provided. An input processing device 202a, reference population database 208, calculator engine 204, and recommender engine 206 are provided by the system 200 as in the system 100.

As with the system 100, the system 200 also comprises a risk assessment engine 222. The risk assessment engine 222 comprises a knowledge base module 224, a risk factor inferencer module 226, and a risk predictor module 228.

The input processing device 202a includes three submodules or applications comprising a genomics data input submodule 202b, a non-genomics data input submodule 202c, and a feedback data input submodule 202d. In various embodiments, data input is done via a web, or mobile application at home, or in an outpatient clinical environment.

The Genomics AI server 202 receives genomics data from various sources via genomics data input submodule 202b that may be integrated with external information providers. In some embodiments, input data may be a file with genotype data uploaded by an individual, by an external genotyping or sequencing service/company using a generic or proprietary application programming interface (API), or by a third party, for example, physicians, dieticians, and aestheticians.

In other embodiments, input data is a file with RNA expression data or protein abundance data. This data may be uploaded by an individual, by an external sequencing service/company using generic or proprietary API, or by a third party, for example, physicians, or dieticians. Genomics data is pre-processed and analyzed using bioinformatics methods.

The Genomics AI server 202 receives non-genomics data from various sources via the non-genomics data input submodule 202c. Non-genomics data may include data about a woman's age, ethnicity, preconception/pregnancy/postpartum stage, demographics, height, weight, activity level, diet, habits, lifestyle, medical history, geolocation, environment, and preferences. Non-genomics data may contain data from physiological tests, for example, blood, urine, and stool, data from wearables, sensors, imaging data from professional devices or smartphones, and other relevant devices.

The non-genomics data input submodule 202c, which may be partially integrated with external information providers, enables input of non-genomics information by generic or proprietary API from imaging devices, sensors, wearables, and other relevant devices, or third-party expert reports, for example, physicians, dieticians, and aestheticians. The non-genomics data input submodule 202c submodule also enables self-reported questionnaires or data input by third parties.

The feedback data input submodule 202d is utilized when the woman provides reviews, survey responses, or other feedback to the system 200 about a dietary program provided to the woman. The feedback data input submodule 202d receives feedback from the woman about specific recipes and food recommendations and likes/dislikes. The feedback data input submodule 202d may also receive reports of adverse effects such as morning sickness, nausea, weight gain during pregnancy or weight loss postpartum, blood pressure, pregnancy complications, baby gestational age, baby weight, and lactation issues.

Upon receipt of genomic and non-genomic data, input processing device 202a shares the received data with a reference population database 208 which is a repository of genomic and non-genomic data for many expectant women and postpartum women as well as women in preconception.

The reference population database 208 may be a component of reference storage 206. Data stored in the reference population database 208 is continuously updated with new entries received from women at various stages of preconception, pregnancy, and postpartum. The reference population database 208 can also be updated by bulk downloads of genomic data from multiple women as well as non-genomic data from third parties, and databases and other repositories not directly associated with the system 200.

Feedback data, received from women at various stages of preconception, pregnancy, or postpartum, or from other parties, is also propagated to the reference population database 208, and, after processing with data normalization engine 218, may also be further transmitted to the calculator engine 204 and recommender engine application 206 to improve assessment of needs and future personalized dietary recommendations, foods, and recipes.

A continuous self-learning system may thereby be set into place. For example, by analyzing via computational algorithms and collected data, the system may infer that women with specific genetic variations are more likely to have more morning sickness in the first trimester if they consume specific foods. Similarly, the system may learn that specific foods and recipes help women deal with morning sickness and nausea.

The reference storage 216 is an information source for computing personalized macro- and micro-nutrient needs and ranges performed by the calculator engine application 204. As with the reference population database 108 of the system 100, the reference population data 208 stored in reference storage 216 and provided by the system 200 may be a single database or multiple databases situated at a single or multiple geographic locations.

The calculator engine 204 comprises a knowledge base application 204a that organizes and dynamically structures state-of-the-art information and data in a knowledge database 204b related to macro- and micro-nutrients effect on the various stages of preconception, pregnancy, and postpartum/lactation. The knowledge database 204b contains data on at least:
(i) levels of macro- and micro-nutrients at different stages of preconception, pregnancy, and lactation/postpartum as recommended, by ob-gyns and nutritionists;
(ii) effects of medical history, age, biometric data, lifestyle factors, dietary restrictions on levels of macro- and micro-nutrients at different stages of preconception, pregnancy, and lactation/postpartum; and
(iii) effects of genetic variations on levels of macro- and micro-nutrients at different stages of preconception, pregnancy, and lactation/postpartum.

The calculator engine 204 receives genomics and non-genomics data from the input processing device 202a. The calculator engine 204 compares the individual genomics and non-genomics data to stored data in the reference population data 208. The calculator engine 204 computes personalized macro- and micro-nutrient risk likelihood predispositions, needs, and ranges based on the received data and knowledge base 204b. The calculator engine 204 may perform the computations of risk likelihood predispositions, needs, and ranges using at least one algorithm that may be proprietary and/or developed by a third-party source. The calculator engine 204 also includes a nutrient calculator 204c that calculates macro- and micro-nutrients for various dietary recommendations and meal plans and furnishes this information to other components of the system 200 as necessary.

The computed macro- and micro-nutrient needs and ranges for the individual woman are transmitted to the recommender engine 206 to generate dietary recommendations, foods, and recipes. The input processing device 202a may transmit additional data collected from the woman to the recommender engine application 206.

In some embodiments, additional data collected from the woman may comprise dietary restrictions, preferences, allergies, and sensitivities. The recommender engine 206 algorithmically generates dietary recommendations, foods, recipes, daily and weekly meal planners, food shopping lists, and dietary tips. These actions may be based on the calculated macro- and micro-nutrient needs and additional data transmitted from the input processing device 202a.

The recommender engine 206 has access to a database of foods and recipes 206b. By performing multi-variable optimization, the recommender engine 206 generates weekly shopping lists and meal plans for a woman based on the woman's ranges for macro- and micro-nutrients, dietary restrictions, food allergies and sensitivities, and personal preferences. The recommender engine 206 relies upon an algorithmic recipe generator 206a to assist in creating recipes.

The database of foods and recipes 206b may have contents contributed, via feedback, by women who have been using the system 200. In embodiments, the recipes and foods are curated, via feedback, by women who have been using the system 200. For example, a specific recipe may be upvoted for women who have morning sickness during the first trimester of pregnancy. The recommender engine 206 and other components have access to a reference storage 216 which hosts the reference population data 208 and an ML discovery module as a self-learning system (not shown in FIG. 2).

Feedback provided by women at various stages of preconception, pregnancy, or postpartum, may be done via a submodule that collects responses from provided dietary recommendations. Specifically, the feedback data may comprise liking/disliking dietary recommendations, foods, and recipes. Feedback data related to dietary recommendations, foods, and recipes are stored in a database of foods/recipes 206b.

The feedback data may then be transmitted to the reference population data 208, and, after processing, be further transmitted to the calculator engine 204 and to the recommender engine 206 to improve future personalized dietary recommendations, foods, and recipes. As noted, a continuous self-learning system may thereby be set in place.

The system 200 also comprises components depicted and enumerated as output/feedback 220a-c which may be equivalent or similar to the user devices 110a-c provided by the system 100 and depicted in FIG. 1. Output/feedback 220a-c receive dietary programs and provide feedback to the Genomics AI server 202. While quantity three output/feedback 220a-c are depicted in FIG. 2 and provided by the system 200, in embodiments more than or less than quantity three output/feedback 220a-c may be provided.

FIG. 2a is a block diagram depicting components of a system 250 according to an embodiment of the present disclosure. The system 250 comprises an input processing device [102], a reference population data [103], a risk assessment engine [104], a calculator engine [105], and a recommender engine [106]. These components correspond to similarly named components of the system 200 depicted in FIG. 2.

The input processing device [102] includes an input device [101] that receives genomics data and non-genomics data from female users. The input processing device [102] consists of three submodules: a genomics data submodule, a non-genomics data input submodule, and a feedback data input submodule. In some embodiments, data input by female users is done via a web, or mobile application at home, or in an outpatient clinical environment.

The input processing device [102] receives genomics data from various sources via the genomics data submodule that is integrated with external information providers. In some embodiments, input data may be a file with genotype data uploaded by an individual, by an external genotyping, or by a sequencing service/company using a generic or proprietary application programming interface (API), or by third parties, for example, physicians, or dieticians. In some embodiments, input data is a file with RNA expression data or protein abundance data. Genomics data (DNA, RNA, or proteomics) is pre-processed and analyzed using bioinformatics methods directed to obtaining quantifiable results to enable further assessments.

The input processing device [102] receives non-genomic data from various sources via the non-genomics data input submodule. Non-genomic data may include data about an individual's age, height, weight, demographics, diet (including allergies, sensitivities, restrictions, and preferences), preconception/pregnancy/postpartum stage, physical activity level, medical history, geolocation, environment, and general concerns. Non-genomic data may contain data from clinical laboratory tests of serum, urine, and stool. Non-genomics data may further contain data from wearables, sensors, and imaging data from professional devices or smartphones, and other relevant devices.

The non-genomics data input submodule, which may be integrated with external information providers, enables input of non-genomics information by generic or proprietary API from relevant devices, including sensors, wearables, imaging devices, or third-party expert reports, for example, physicians, and dieticians. The non-genomics data input submodule also enables self-reported questionnaires or data input by third parties.

The feedback data input submodule is utilized when the user receives output from the system regarding optimal levels of macro- and micronutrients, food, and recipe recommendations. In some embodiments, the feedback data input submodule is used by the user to report data related to specific recipes, food recommendations, and likes/dislikes. The feedback data input submodule may also be used by the user, or the third party, for example a physician, to report adverse effects such as morning sickness, nausea, food cravings, weight gain during pregnancy, or weight loss postpartum, blood pressure, pregnancy complications, baby gestational age, baby weight, and lactation issues.

Upon receipt of genomic and non-genomic data, the input processing device [102] propagates the received data to reference storage [103] which is a repository of genomic and non-genomic data for a plurality of individual females. The data stored in reference storage [103] is continuously updated with new entries received from individuals via the input processing device [102]. Reference storage [103] can also be updated by bulk downloads of genomic data from multiple individual females as well as non-genomic data from external sources, data repositories, and third parties.

Feedback data, received from the user, is propagated to the reference storage [103]. After processing, using various data analysis tools, the feedback data is provided to the risk assessment engine [104], the calculator engine [105], and the recommender engine [106], to improve predictive models for assessment of the risk predisposition to gestational diabetes.

Based on processing by at least the risk assessment engine [104], the feedback data may be used to further optimize and fine-tune dietary and lifestyle recommendations. A continuous self-learning system may thereby be set into place. By analyzing collected data, via machine learning methodologies, the system may improve the predictive model for assessment of the risk predisposition to gestational diabetes.

The system may further build predictive models and assess risk predispositions to other pregnancy-related complications and side-effects. The system may further infer that women with specific combinations of genetic variations, for example, those that are associated with higher levels of clinically measured biomarkers, such as glucose or leptin, are more likely to develop gestational diabetes if they consume specific foods. Similarly, the system may learn that specific foods, for example, oily fish, or lifestyle changes may assist at-high-risk women to lower their chances of gestational diabetes.

The reference storage [103] may provide bases for computing, via a machine learning methodology, a risk predisposition to gestational diabetes for an individual female performed by the risk assessment engine [104]. Reference storage [103] may further provide bases for computing personalized macro- and micro-nutrient predisposition risk likelihoods and needs performed by the calculator engine [105].

The risk assessment engine [104] consists of three modules: a knowledge base module, a risk factor inferencer module, and a risk predictor module. The knowledge base module integrates genomic and non-genomic data from the reference storage [103] with external knowledge on gestational diabetes from published studies, randomized trials, and data repositories. The knowledge base module transforms this heterogeneous data from different sources into structured data using suitable computational methodologies and natural language processing tools.

The risk factor inferencer module infers, by applying various machine learning methodologies, from at least the knowledge base module, associations between various genomic and non-genomic factors and the risk of gestational diabetes. Risk factors include dietary intakes, levels of clinically measured biomarkers and metabolites, physical activity, sleep, environmental factors, for example, pollution, and other phenotypic traits, for example, body mass index (BMI). The risk factor inferencer module computes, via at least a method of Mendelian randomization, genetic-based risk factors for gestational diabetes utilizing the data from at least the reference storage [103]. The risk factor inferencer module further validates inferred and genetically constructed risk factors of gestational diabetes on the data from the reference storage [103].

The risk predictor module computes risk predisposition likelihoods to gestational diabetes for women by integrating genomic and non-genomic data and inferred and computed risk factors in a machine learning model. The risk predictor may perform the computations of risk predisposition using at least one algorithm that may be proprietary and/or developed by a third-party source, for example, polygenic risk scores.

In an embodiment, the risk predictor module applies a supervised machine learning model to integrate genomic and non-genomic data from the reference storage [103]. The risk predictor module uses best practices of machine learning to develop and validate risk predisposition predictions utilizing the cases and controls for gestational diabetes. In some embodiments, the machine learning model can be a generalized linear model, a classification model, a Bayesian model, a Neural Network Analysis (NNA), or an ensemble of several models.

The risk predictor module optimizes predictions for risk predisposition to gestational diabetes. It further calculates, via a suitable computational methodology, a proportion of risk from genomics, diet, and lifestyle factors.

In one example, the risk factor inferencer module integrated publicly available summary statistics data on gestational diabetes from the UK Biobank and several large-scale independent metabolomic studies. By applying Mendelian randomization (MR) computational methodology and/or at least one machine learning methodology to this data, the risk factor inferencer module determined that genetically constructed levels of pre-pregnancy waist-to-hip ratio (WHR), BMI, adiposity, or abdominal fat deposition are significantly correlated with the risk of gestational diabetes. (Peršić M M, Vladimir K, Karpov S, Štorga M, Mostashari A, Khanin R. Polygenic Risk Score and Risk Factors for Gestational Diabetes. J Pers Med. 2022 Aug. 26; 12(9):1381. doi: 10.3390/jpm12091381. PMID: 36143166; PMCID: PMC9505112.) Specifically, higher WHR increases the risk predisposition to gestational diabetes by nearly a factor of two. Additionally, MR identified that genetically constructed levels of glucose, glycated hemoglobin, and leptin each increase the risk of gestational diabetes by more than a factor of three. Further, higher genetically constructed levels of liver enzyme alanine aminotransferase increase the risk of gestational diabetes by 30%. Additionally, high levels of several metabolites, including acylcarnitines, increase the odds of gestational diabetes by a factor of two.

On the other hand, the MR results indicate that higher genetically proxied levels of healthy fats (mono-unsaturated fatty acids, omega-7, omega-9) significantly reduce the risk of gestational diabetes by a factor of two.

These results further underscore the importance of maintaining a healthy BMI/WHR, low adiposity, and abdominal fat deposition, maintaining normal glucose levels, and consuming healthy fats. Furthermore, these results provide a roadmap for identifying risk factors of gestational diabetes, developing practical tests to measure risk-elevated biomarkers, and most importantly early screening that identifies women at higher risk of gestational diabetes at the preconception or early pregnancy stages before its onset allowing comprehensive monitoring and preventative programs to mitigate the risks.

The calculator engine [105] receives genomics and non-genomics data from at least the reference population module [103]. The calculator engine [105] comprises two modules: a knowledge base of nutrients and a nutrient calculator. The knowledge base module organizes and dynamically structures state-of-the-art knowledge related to macro and micronutrients at each stage of preconception, pregnancy, and postpartum/lactation.

The knowledge base of nutrients contains data on
(i) levels of micro- and macronutrients required at different stages of preconception, pregnancy, and lactation/postpartum as recommended by obstetrician-gynecologists and nutritionists;
(ii) effects of medical history, age, biometric data, lifestyle factors, dietary restrictions on levels of micro and macronutrients required at different stages of preconception, pregnancy, and lactation/postpartum; and
(iii) effects of genetic variations on levels of micro- and macronutrients that are required at different stages of preconception, pregnancy, and lactation/postpartum.

The knowledge base further comprises data on:
(i) micro- and macronutrients that may decrease the risk of gestational diabetes (e.g., vitamin D, mono-unsaturated fats);
(ii) micro- and macro-nutrients that may elevate the risk of gestational diabetes (e.g., glucose, unhealthy fats);
(iii) biomarkers that are associated with an elevated risk of gestational diabetes; and biomarkers that are associated with lower risk of gestational diabetes;
(iv) micro- and macro-nutrients and foods that affect levels of risk-elevating biomarkers for gestational diabetes, for example, leptin;
(v) micro- and macro-nutrients that affect levels of risk-decreasing biomarkers for gestational diabetes (e.g. adiponectin).

The nutrient calculator of the calculator engine 204c computes personalized micro- and macro-nutrient predisposition risk likelihoods, needs, and ranges based on the received data of an individual, and the knowledge base, by comparing the individual genomics and non-genomics data to the reference population data.

A predisposition risk likelihood of a specific macro- or micro-nutrient reflects levels of a said nutrient relative to the reference population, indicating either a risk of deficiency of the specific nutrient, for example, Iron deficiency, or an overload of the said nutrient, for example iron. The calculator engine [105] may perform computations of predisposition risks, needs, and ranges using at least one algorithm that may be proprietary and/or developed by a third-party source, for example, a polygenic risk score.

The computed macro- and micro-nutrient needs and ranges for the individual woman are transmitted to the recommender engine [106] to generate dietary recommendations, foods, and recipes. The input processing device [102] may transmit additional data collected from the user to the recommender engine [106]. In some embodiments, additional data collected from the user may comprise dietary restrictions, preferences, allergies, and sensitivities. The recommender engine [106] algorithmically generates dietary recommendations, foods, recipes, daily and weekly meal planners, food shopping lists, and dietary tips. These actions are based on the calculated personalized macro- and micro-nutrient needs and additional data transmitted from the input processing device [102].

In an embodiment, the recommender engine [106] has access, via API, to a large database of foods and recipes. By performing multi-variable optimization, the recommender engine [106] generates daily/weekly shopping lists and meal plans for individual women based on personalized risk predispositions to gestational diabetes, ranges for macro- and micro-nutrients, dietary restrictions, food allergies and sensitivities, and personal preferences.

In other embodiments, the recommender engine [106] has a proprietary database of recipes, contributed, via feedback, by women who have been using the system. In embodiments, the recipes and foods are curated, via feedback, by women who have been using the system. For example, a specific recipe may be upvoted for women who have morning sickness during the first trimester of pregnancy.

Feedback may be provided by output/feedback [107] that collects responses from an individual woman on the provided dietary recommendations, shopping lists, and meal plans. Specifically, the feedback data may comprise liking/disliking dietary recommendations, foods, and recipes. Feedback data related to dietary recommendations, foods, and recipes are stored in the knowledge base of the recommender engine [106]. The feedback data may then be transmitted to reference storage [103], and, after processing, be further transmitted to the calculator engine [105] and to the recommender engine [106] to improve future personalized dietary recommendations, foods, and recipes. A continuous self-learning system may thereby be set in place.

In an embodiment, the recommender engine [106] may execute on a mobile computing device such as a smartphone or a tablet computing device. The recommender engine [106] hardware platform may be a desktop computing device or a laptop computing device. The recommender engine [106] hardware platform may include more than one computing device, such as output/feedback [107], configured to provide a user interface and one or more server computing devices configured to provide computational functionality such as the functionality of the calculator engine [105]. In such embodiments, the user computing device and one or more server computing devices may communicate via any suitable communication technology or technologies. Such technologies may comprise a wired technology comprising Ethernet, USB, or the Internet, or a wireless technology comprising WiFi, WiMAX, 3G, 4G, LTE, or Bluetooth.

In an embodiment, a system for computing risk predisposition to gestational diabetes mellitus for an individual woman is provided. The system comprises a computer and an application stored in the computer that when executed computes, based on received data, risk predisposition to gestational diabetes for a female. The system also calculates, based at least on the computed risk predisposition, calories and macro- and micro-nutrient needs for the female. The system also generates, based on the calculations, dietary recommendations, foods, and recipes for the female.

The system further receives feedback from a plurality of female humans regarding liking/disliking and adverse reactions comprising at least one of morning sickness and nausea. The system further receives additional data comprising at least one of pregnancy complications, blood pressure, and glucose levels. The system further improves, based at least on the feedback and the received additional data, and via at least a machine learning methodology, assessment of risk predisposition to gestational diabetes for the plurality of female humans.

The received data comprises genomics and non-genomic data describing women. The system performs the computations via at least one machine learning methodology. Longitudinal data comprising at least the additional data is used to discover associations and to build, via at least one of the machine learning methodologies, predictive comprehensive models for at least one of pregnancy-related traits and pregnancy complications.

The discovery of associations and the building of the models accounts for genomics and non-genomics data comprising at least one of dietary consumption and lifestyle during preconception, pregnancy, and postpartum stages.

The system presents inquiries to the female in at least a questionnaire format, the inquiries including questions directed to at least one of liking/disliking recommended foods and recipes, adverse reactions, and questions related to one stage of preconception, pregnancy, or postpartum/lactation. The longitudinal data further comprises responses to inquiries regarding potential gestational diabetes development.

In another embodiment, a system for determining risk predisposition to gestational diabetes for a female is provided. The system comprises a computing device and an application executing on the computing device that receives feedback data reported by a plurality of users, the feedback related to specific food and dietary recommendations associated with at least mitigation of gestational diabetes. The system also receives user data comprising genomics data and non-genomics data from the plurality of users. The system also provides the feedback data and the user data to a risk assessment engine. The system also directs the risk assessment engine to adjust, based at least on the feedback data and the user data, predictive models for assessment of risk predisposition to gestational diabetes. The system also builds a continuous self-learning system that further refines calculations of risk predispositions based on ongoing adjustments of the predictive models, and continuing receipt of feedback data and user data.

The feedback data further describes adverse reactions comprising at least one of morning sickness, nausea, food cravings, weight gain during pregnancy, weight loss post-partum, blood pressure, pregnancy complications, baby gestational age, baby weight, and lactation issues.

By analyzing collected data, via at least one machine learning methodology, the system supports the building of the continuous self-learning system.

The genomics data comprises at least one of DNA data, RNA expression data, protein abundances data, measured by genotyping array, or next-generation sequencing. The non-genomics data comprises at least one of age, height, weight, ethnicity, medical history, diet, demographics, and stage, wherein stage comprises one of preconception, pregnancy, and postpartum.

In yet another embodiment, a method of determining genomics-based risk factors of gestational diabetes is provided. The method comprises a risk factor inferencer module of a risk assessment engine inferring associations between genomic and non-genomic factors and risk of gestational diabetes. The method also comprises the module, via at least a method of Mendelian randomization, and based at least on the inferred associations, computing genetic-based risk factors for gestational diabetes, using data from at least a reference storage. The method also comprises the module validating the inferred associations and validating genetic-based risk factors of gestational diabetes.

The method further comprises a risk predictor module of the engine computing risk predisposition likelihood to gestational diabetes for individual women by integrating genomic and non-genomic data and inferred and computed risk factors using a machine learning model.

The method further comprises the risk predictor module performing the computations using at least one algorithm, the algorithm at least one of proprietary and developed by a third-party source.

The method further comprises the risk predictor module applying a supervised machine learning model to integrated female data, the model comprising at least one of a generalized linear model, a classification model, a Bayesian model, or a Neural Network Analysis (NNA).

The method further comprises the risk predictor module calculating, via computational methodology, a proportion of risk from at least one of genomics, diet, and lifestyle factors.

The method further comprises a knowledge base module of the engine gathering heterogeneous information by integrating genomic and non-genomic data from the reference storage with external knowledge about gestational diabetes, the external knowledge comprising at least one of published studies, randomized trials, and data repositories.

The method further comprises the knowledge base module transforming the heterogeneous data into structured data using computational methodologies and natural language processing tools.

In embodiments, general steps of systems and methods provided herein may comprise:
1. The system receives an individual woman's genomics data and non-genomics data.
2. The system adds the woman's data to the population data and compares the woman's data to the population data.
3. The system collects longitudinal data on pregnancy progress and lactation that includes objective measurements and self-reported data from individual women. Objective data measurements include an individual woman's weight, age, medical data, physiological data, and wearables/sensors data measured at various time intervals. Objective data may contain reports from physicians and results from medical laboratories or at-home tests for pregnancy-related biomarkers. Self-reported data includes an individual woman's pregnancy complications, including gestational diabetes, gestational hypertension, or preeclampsia, symptoms (such as morning sickness, heartburn, nausea, and vomiting), food cravings, food intolerances, and sensitivities, liking or disliking specific foods and recipes, emotional wellbeing at various time-points.
4. The system integrates population genomics data with the population's non-genomics data.
5. The system propagates the individual woman's longitudinal data to the module with population data.
6. The system computes the risk likelihood (risk) predisposition to gestational diabetes for the individual woman by utilizing machine learning methods and comparing an individual woman's data to the population data.
7. The system computes the individual woman's macro- and micro-nutrient needs and ranges based on pregnancy/lactation stage, calculated risk predisposition to gestational diabetes, activity level, age, genomics data, medical history, and physiological and other data by utilizing machine learning methods and comparing an individual's woman's data to the population data.
8. The system algorithmically generates personalized weekly shopping lists and meal plans by performing multi-variable optimization of food and recipe databases for the individual woman. These steps are based on her personalized ranges for macro- and micro-nutrients, dietary restrictions, food allergies and sensitivities, and personal preferences.
9. The system relies on a reporting and feedback module to send and receive data.

What is claimed is:

1. A system for computing risk predisposition to gestational diabetes mellitus for an individual woman comprising:
a computer; and
an application stored in the computer that when executed:
computes, based on received data, risk predisposition to gestational diabetes for a female,
calculates, based at least on the computed risk predisposition, calories and macro- and micro-nutrient needs for the female, and
generates, based on the calculations, dietary recommendations, foods, and recipes for the female.

2. The system of claim 1, wherein the system further:
receives feedback from a plurality of female humans regarding liking/disliking and adverse reactions comprising at least one of morning sickness and nausea,
receives additional data comprising at least one of pregnancy complications, blood pressure, and glucose levels,
improves, based at least on the feedback and the received additional data, and via at least a machine learning methodology, assessment of risk predisposition to gestational diabetes for the plurality of female humans.

3. The system of claim 1, wherein the received data comprises genomics and non-genomic data describing women.

4. The system of claim 1, wherein the system performs the computations via at least one machine learning methodology.

5. The system of claim 1, wherein longitudinal data comprising at least the additional data is used to discover associations and to build, via at least one of the machine learning methodologies, predictive comprehensive models for at least one of pregnancy-related traits and pregnancy complications.

6. The system of claim 5, wherein the discovery of associations and the building of the models accounts for genomics and non-genomics data comprising at least one of dietary consumption and lifestyle during preconception, pregnancy, and postpartum stages.

7. The system of claim 1, wherein the system presents inquiries to the female in at least a questionnaire format, the inquiries including questions directed to at least one of liking/disliking recommended foods and recipes, adverse reactions, and questions related to one stage of preconception, pregnancy, or postpartum/lactation.

8. The system of claim 5, wherein the longitudinal data further comprises responses to inquiries regarding potential gestational diabetes development.

9. A system for determining risk predisposition to gestational diabetes for a female, comprising:
a computing device; and
an application executing on the computing device that:
receives feedback data reported by a plurality of users, the feedback related to specific food and dietary recommendations associated with at least mitigation of gestational diabetes,
receives user data comprising genomics data and non-genomics data from the plurality of users,
provides the feedback data and the user data to a risk assessment engine,
directs the risk assessment engine to adjust, based at least on the feedback data and the user data, predictive models for assessment of risk predisposition to gestational diabetes, and
builds a continuous self-learning system that further refines calculations of risk predispositions based on ongoing adjustments of the predictive models, and continuing receipt of feedback data and user data.

10. The system of claim 9, wherein the feedback data further describes adverse reactions comprising at least one of morning sickness, nausea, food cravings, weight gain during pregnancy, weight loss post-partum, blood pressure, pregnancy complications, baby gestational age, baby weight, and lactation issues.

11. The system of claim 9, wherein by analyzing collected data, via at least one machine learning methodology, the system supports the building of the continuous self-learning system.

12. The system of claim 9, wherein the genomics data comprises at least one of DNA data, RNA expression data, protein abundances data, measured by genotyping array, or next-generation sequencing.

13. The system of claim 9, wherein the non-genomics data comprises at least one of age, height, weight, ethnicity, medical history, diet, demographics, and stage, wherein stage comprises one of preconception, pregnancy, and postpartum.

14. A method of determining genomics-based risk factors of gestational diabetes, comprising:
a risk factor inferencer module of a risk assessment engine inferring associations between genomic and non-genomic factors and risk of gestational diabetes;
the module, via at least a method of Mendelian randomization, and based at least on the inferred associations, computing genetic-based risk factors for gestational diabetes, using data from at least a reference storage; and
the module validating the inferred associations and validating genetic-based risk factors of gestational diabetes.

15. The method of claim 14, further comprising a risk predictor module of the engine computing risk predisposition likelihood to gestational diabetes for individual women by integrating genomic and non-genomic data and inferred and computed risk factors using a machine learning model.

16. The method of claim 15, further comprising the risk predictor module performing the computations using at least one algorithm, the algorithm at least one of proprietary and developed by a third-party source.

17. The method of claim 14, further comprising the risk predictor module applying a supervised machine learning model to integrated female data, the model comprising at least one of a generalized linear model, a classification model, a Bayesian model, or a Neural Network Analysis (NNA).

18. The method of claim 14, further comprising the risk predictor module calculating, via computational methodology, a proportion of risk from at least one of genomics, diet, and lifestyle factors.

19. The method of claim 14, further comprising a knowledge base module of the engine gathering heterogeneous information by integrating genomic and non-genomic data from the reference storage with external knowledge about gestational diabetes, the external knowledge comprising at least one of published studies, randomized trials, and data repositories.

20. The method of claim 19, further comprising the knowledge base module transforming the heterogeneous data into structured data using computational methodologies and natural language processing tools.
